SEQUENCE LISTING (2 sequences)

SEQ ID NO: 1 - protein, 227 aa, homo sapiens
DLQLAAELGKTLLDRNTELEDSVQQMYTTNQEQLQEIEYLTKQVELLRQMNEQHAKVYEQLDVTARELEETNQKLVADSKASQQKILSLTETIECLQTNIDHLQSQVEELKSSGQGRRSPGKCDQEKPAPSFACLKELYDLRQHFVYDHVFAEKITSLQGQPSPDEEENEHLKKTVTMLQAQLSLERQKRVTMEEEYGLVLKENSELEQQLGATGAYRARALELEAE

SEQ ID NO: 2 - protein, 76 aa, homo sapiens
MNEQHAKVYEQLDVTARELEETNQKLVADSKASQQKILSLTETIECLQTNIDHLQSQVEELKSSGQGRRSPGKCDQ

(19) United States
(12) Patent Application Publication
(10) Pub. No.: US 2003/0004092 A1
(43) Pub. Date: Jan. 2, 2003

(54) Methods and agents for inducing apoptosis and methods for their identification

(76) Inventors: Robert B. Darnell, Pelham, NY (US); John P. Corradi, Washington, DC (US); Woong-Yang Park, Seoul (KR)

Correspondence Address: David A. Jackson, Esq., KLAUBER & JACKSON, 411 Hackensack Avenue, Hackensack, NJ, 07601, US

(21) Appl. No.: 09/923,284

(22) Filed: Aug. 6, 2001

Related U.S. Application Data
(63) Continuation of application No. 09/174,913, filed on Oct. 19, 1998, now abandoned.

(51) Int. Cl. A61K 31/00; A61K 31/56

(52) U.S. Cl. 514/1; 514/169

(57) ABSTRACT

Agents and method are described for inducing apoptosis in normal and diseased cells by antagonizing the interaction between onconeural antigens and apoptosis-inducing proteins, such as transcription factors. The antagonism of the interaction leads to increased bioavailability of the protein, which then directly or indirectly induces cell cycle genes. In dysproliferative cells, such gene activation and induction of cell cycle genes leads to apoptosis and death of dysproliferative cells. Therapies are directed to the treatment of diseases such as cancer. Normal cells, such as germ cells, may be treated by the agents to undergo apoptosis, for example, in the induction of sterility. Methods for the identification of suitable agents of the present invention are described by determining the extent of interference of binding between onconeural antigens and their fragments and apoptosis-inducing proteins and their fragments, in cell-free and cell-based assays.

### FIELD OF THE INVENTION

The present invention relates to agents and method for inducing apoptosis in normal or diseased cells by antagonizing the interaction between onconeural antigens and apoptosis-inducing proteins, and methods for the identification of agents which antagonize said interaction.

### BACKGROUND OF THE INVENTION

The molecular basis underlying dysproliferative diseases such as cancer is the dysregulation of gene transcription mediated by aberrant levels or inappropriate interactions among transcription factors, which in turn act in complex pathways to activate cell cycle genes. One particular family of transcription factors implicated in oncogenesis is the Myc family of transcription factors, whose dysregulation is implicated in oncogenesis. Excessive levels of active transcription factor are believed to activate cell cycle genes and induce capable cells to enter the cell cycle (Bouchard et al., 1990); cells which cannot successfully enter the cell cycle upon such activation undergo apoptosis and die, such as neuronal cells. A recent study (Ulrich et al., 1998) suggests that Myc itself is capable of mediating neuronal cell apoptosis. The role of the c myc oncogene in cell growth and apoptosis has been reviewed by Evans & Littlewood (1993).

Paraneoplastic neurologic disorders are immune-mediated neuronal degenerations that develop in the setting of malignancy. They provide perhaps the clearest examples of naturally-occurring tumor immunity in man. It is believed that the disorders are initiated when neuron-specific proteins, normally immunologically privileged, are ectopically expressed in tumor cells and are thereby recognized as foreign (tumor) antigens. Such patients are asymptomatic until this immune response becomes competent to recognize antigens expressed in the nervous system, leading to an immune-mediated neuronal death. These rare disorders thus touch on important questions of tumor immunity, autoimmune neurologic disease, and neuron-specific protein function. Paraneoplastic cerebellar degeneration (PCD) is one of the best-studied PNDs. Characterization of fifty-five PCD patients (Peterson et al., 1992) revealed that they almost invariably have breast or ovarian cancer, and that their cerebellar degeneration is characterized pathologically by Purkinje cell death. The immune response in PCD is characterized by high titers of serum and cerebrospinal fluid antibodies that recognize a ˜55 kDa antigen in the patient's tumors and in cerebellar Purkinje neurons. PCD antisera has been used to clone cDNAs encoding several related target antigens (Dropcho et al., 1987; Sakai et al., 1990; Fathallah-Shaykh et al., 1991), only one of which, cdr2, is expressed in PCD tumor specimens (Corradi et al., 1997). The cdr2 gene is widely transcribed, but the protein has only been found to be expressed in cerebellar Purkinje neurons, some brainstem neurons, and spermatogonia (Corradi, et al., 1997), all immune-privileged sites. The major insight to the biologic function of cdr2 has been the identification of structural motifs in the predicted amino acid sequence. The amino-terminal 150 amino acids of cdr2 contain an acidic region of 30 amino acids, followed by an extended amphipathic helix of 100 amino acids and a classical helix-leucine zipper (HLZ) dimerization motif (Fathallah-Shaykh et al., 1991). The antigen was found to be localized to the cytoplasm, where it can be found both free and associated with membrane-bound ribosomes (Hida et al., 1994).

Myc is a member of a group of transcription factors that are responsible for gene activation and the entry of quiescent cells into the cell cycle; inappropriate expression or increased bioavailability of Myc appears to be responsible for oncogenesis and the promotion of dysproliferative diseases. A variety of proteins have been described that bind to and regulate Myc transcriptional activity in the nucleus. The canonical Myc binding protein is Max, which binds nuclear Myc via its HLZ domain to mediate transcriptional activation (Blackwood et al., 1992). Other nuclear Myc binding partners have been described that bind outside of the HLZ, including TRRAP, which interacts with the Myc N-terminal domain (McMahon et al., 1998), and YY1 (Shrivastava et al., 1993). In addition, a series of HLZ proteins, including Mad1, Mxi1, Mad3, Mad4 and Mnt (Ayer et al., 1993; Hurlin et al., 1996; Hurlin et al., 1997) down regulate Myc activity indirectly, by competing with nuclear Myc for binding to Max. There have been previous reports that Myc protein can be detected in the cytoplasm of certain cell types. C-Myc has been previously detected in the cytoplasm of cell lines (Craig, et al., 1993) and tumor samples (Lipponen, 1995; Pietilainen et al., 1995; Boni et al., 1998). In neurons, the detection of Myc in Purkinje cell cytoplasm parallels the observation that N-Myc, which is expressed in the nuclei of cerebellar Purkinje neurons during development, but localizes to Purkinje cell cytoplasm in adults (Wakamatsu et al., 1993).

However, no proteins that directly bind to and inhibit Myc activity have been described.

It is towards the identification of new agents and methods of inducing apoptosis in normal or diseased cells for the treatment of various conditions and disorders, such as dysproliferative diseases, and the specific destruction of certain types of cells, such as neoplastic and germ cells, that the present invention is directed.

The citation of any reference herein should not be construed as an admission that such reference is available as “Prior Art” to the instant application.

### SUMMARY OF THE INVENTION

The present invention relates to a method for inducing apoptosis of cells in the body by administering a therapeutically-effective amount of an agent which is capable of antagonizing the interaction between an onconeural antigen and an apoptosis-inducing protein. Non-limiting examples of conditions in which apoptosis of cells is desired therapeutically and may be achieved by the by the agents and methods of the present invention include dysproliferative diseases such as cancer, and germ cells, whose destruction will bring about sterility. Non-limiting examples of apoptosis-inducing factors include transcription factors, such as those of the Myc family of oncoproteins, as well as other proteins which interact with onconeural antigens. Apoptosis-inducing transcription factors are preferred, such as N-Myc and C-myc. Among dysproliferative diseases, treatment of gynecological cancers is preferred, and most preferably ovarian and breast cancer. The onconeural antigens include but are not limited to cdr2, cdr3, Nova, Hu, or amphiphysin; cdr2 is preferred.

The present invention further related to agents useful for the treatment of various conditions in which apoptosis of certain cells is desired, such as cells in individuals with dysproliferative diseases including cancer, and normal cells, such as germ cells for the induction of sterility. Non-limiting examples of agents suitable for the practice of the present invention include antibodies or antibody fragments capable of interfering with the interaction between onconeural antigens and apoptosis-inducing proteins. Antibodies of the present invention include those which are bind to onconeural antigen such as, but not limited to, cdr2, cdr3, Nova, Hu, and amphiphysin. Preferably, the antibody or fragment thereof binds to cdr2.

In a further embodiment, agents of the present invention are those which interact with the HLZ region of the onconeural antigen or the apoptosis-inducing factor. Such HLZ region-interacting molecules may include, but are not limited to, fragments of onconeural antigens and proteins bearing the HLZ region. Non-limiting examples include polypeptide fragments of cdr2 comprising amino acids 16 through 192 (SEQ ID NO:1) and amino acids 65-140 of cdr2 (SEQ ID NO:2).

The present invention further relates to a method for promoting apoptosis of cells comprising contacting said cells with an effective amount of an agent capable of antagonizing the interaction between an onconeural antigen and an apoptosis-inducing protein. Conditions treatable by the agents and methods of the present invention include dysproliferative diseases such as cancer and psoriasis, preferably gynecological cancers, and most preferably ovarian and breast cancer. Treatment of nromal cells is also an embodiment of the present invention. Induction of apoptosis in germ cells achieves sterility. The onconeural antigens include but are not limited to cdr2, cdr3. Nova, Hu, or amphiphysin; cdr2 is preferred. The apoptosis-inducing factors include but are not limited to transcription factors such as those in the myc family; N-Myc and C-myc are preferred.

The present invention further related to agents useful for promoting apoptosis of cells. Non-limiting examples of agents suitable for the practice of the present invention include antibodies or antibody binding fragments capable of interfering with the interaction between onconeural antigens and apoptosis-inducing proteins. Antibodies of the present invention include those which are bind to onconeural antigen such as, but not limited to, cdr2, cdr3, Nova, Hu, and amphiphysin. Preferably, the antibody or fragment thereof binds to cdr2.

In a further embodiment, agents of the present invention useful for promoting apoptosis are those which interact with the HLZ region of the onconeural antigen or the apoptosis-inducing factor. Such HLZ region-interacting molecules may include, but are not limited to, fragments of onconeural antigens and proteins bearing the HLZ region. Non-limiting examples include polypeptide fragments of cdr2 comprising amino acids 16 through 192 (SEQ ID NO:1) and amino acids 65-140 of cdr2 (SEQ ID NO:2).

The invention herein also relates to methods for identifying an agent capable of promoting apoptosis by antagonizing the interaction between an onconeural antigen and an apoptosis-inducing protein. The methods comprising the steps of (1) preparing a mixture comprising an onconeural antigen or a fragment thereof and an apoptosis-inducing transcription factor or a fragment thereof; (2) contacting said mixture with an agent being evaluated for its ability to antagonize the interaction between said onconeural antigen and said apoptosis-inducing transcription factor; (3) evaluating the extent of interference by said agent of the interaction between said onconeural antigen and said apoptosis-inducing transcription factor; and (4) determining from said extent of interference the capability of said agent to interfere with said interaction. The onconeural antigen or fragment, or the transcription factor, or fragment may be modified to include a polypeptide sequence for aid in binding or identifying the interactions. Non-limiting examples of methods for assessing the interference in binding between these molecules include (1) determining the extent of binding, for example, by electrophoretic means, examples of which include a GST pull-down assay or coprecipitation assay; (2) assessing the transcriptional activity in a assay, for example, employing a reporter gene or genes; (3) evaluating the effect of an agent on the subcellular distribution of the transcription factor by immunochemical localization or subcellular fractionation means; and (4) assessing the effect of an agent on cell death. Preferred components in the above-described assays include, as onconeural antigens, cdr2, cdr3, Nova, Hu, or amphiphysin; most preferred is cdr2. Examples of apoptosis-inducing proteins for use in the assay include transcription factors, preferably those in the myc family, and most preferably, N-Myc and C-myc. In a further example of a method for identifying agents which induce apoptosis by interfering with the aforementioned interaction, an assay for cell death may be employed.

These and other aspects of the present invention will be better appreciated by reference to the following drawings and Detailed Description.

### BRIEF DESCRIPTION OF THE DRAWINGS

FIG. 1 demonstrates that cdr2 and Myc interact in vitro, using a GST pull-down assay in which immobilized GST fusion proteins were incubated with full length in vitro translated Myc and cdr2. The arrows indicate the size of the full-length ³⁵S-labeled proteins. Panel A shows that full length ³⁵S-labeled Myc binds to GST-cdr2 in vitro. Panel B shows that full-length ³⁵S-labeled cdr2 interacted with a truncated Myc fusion protein that contained the HLZ domain (Myc439) and also bound to itself (GST-cdr2).

FIG. 2 depicts the immunohistochemical colocalization of cdr2 and Myc in the cytoplasm of rat cerebellar Purkinje neurons. Panel A shows a section of adult rat cerebellar cortex stained with PCD CSF which reveals cdr2 immunoreactivity in the cytoplasm of Purkinje cells. The three cellular layers of the cerebellar cortex are indicated as the molecular (M) layer, the Purkinje (P) cell layer, and the granule (G) cell layer. Panel B shows a serial section stained with anti-Myc mouse monoclonal antibody shows the same localization. Insets show cytoplasmic staining of cdr2 and Myc, sparing the nucleus. Panel C shows a serial section stained with POMA [please define] CSF as a control to demonstrate that these immunohistochemical conditions preserved nuclear reactivity to the Nova protein.

FIG. 3 demonstrates that cdr2 co-immunoprecipitates with c-Myc in mouse cerebellum. Mouse cerebellar homogenate was precipitated with normal rabbit sera (lane 2), anti-Myc rabbit polyclonal antibody (lane 3) or anti-cdr2 PCD patient's CSF (lane 4). Immune complexes were analyzed by western blot using PCD (anti-cdr2) patient's serum. Lane 1 is a Western blot of the cerebellar lysate. Two cdr2 immunoreactive proteins are indicated, the lower of which may preferentially be co-immunoprecipitated by Myc (lane 3).

FIG. 4 demonstrates that cdr2 co-immunoprecipitates with N-Myc in mouse cerebellum, in a similar fashion to that described in FIG. 3.

FIGS. 5 and 6 depict an experiment which demonstrates that cdr2 represses Myc transcriptional activity. Error bars indicate two standard deviations. In FIG. 5, rat 1A fibroblasts were transiently transfected with reporter plasmids, then were co-transfected with either no additional plasmid (−), a Myc expressing plasmid (Myc) or Myc together with a cdr2 expressing plasmid (Myc+cdr2). Transfection with Myc alone resulted in an average 2.6-fold induction of CAT activity. Cotransfection with cdr2 inhibited the Myc induced CAT activity to near baseline levels. In FIG. 6, NIH3T3 cells were transiently transfected with reporter plasmids, a Myc expressing plasmid, and the indicated amount of cdr2-expressing plasmid. Cotransfected cdr2 inhibited Myc-dependent reporter gene activity in a titratable manner. The results shown represent the average transfections performed in triplicate.

FIGS. 7 and 8 depict the co-localization of cdr2 and Myc in the cytoplasm of a cdr2-inducible cell line. In FIG. 7, HTC-75 cells were incubated for 72 hrs in the presence (control; cdr2 expression off) or absence (cdr2; cdr2 expression on) of doxycyclin. Fixed cells were stained with affinity purified cdr2 antibodies (green) and Myc monoclonal antibodies (red) and examined by confocal microscopy (Zeiss). Colocalization was examined by a combination of red and green fluorescence (yellow). In FIG. 8, Myc is shown to coprecipitate with T7-tagged cdr2 in HTC-75 cells. Expression of T7-tagged cdr2 protein was induced by removing doxycyclin from the media. Cell extracts were run on Western blot before immunoprecipitation with the indicated antibodies. Blots were then probed with monoclonal antibodies to Myc or the T7-tag, as indicated.

FIG. 9 demonstrates the inhibition of interactions between cdr2 and Myc by PCD patients' sera using GST pull-down assays. Washed sera obtained from six different PCD patients or non-PCD control sera were mixed with in vitro translated ³⁵S-methionine labeled Myc protein. Specifically bound Myc protein was assessed by SDS-PAGE and fluorography. Quantitation of the ratio of cdr2 to Max protein precipitated in the presence of each serum sample indicated that PCD sera inhibited ³⁵S-Myc pull downs by an average of 5.5 (range 4.9-7.1) fold relative to the average affect of non-PCD sera.

FIG. 10 compares the histopathology of CN XII motor neurons in Nova-1 null (knockout) mices compared to wild-type mice, revealing shows selective degeneration of motor neurons in the hypoglossal nucleus of the Nova-1 null mice.

### DETAILED DESCRIPTION OF THE INVENTION

It has been discovered by the inventors herein that a normal cellular function of onconeural antigens is the prevention of apoptosis by binding to apoptosis-promoting proteins within the cytoplasm. This surprising and unanticipated discovery that onconeural antigens protect the cell from apoptosis by binding to apoptosis-inducing factors, reducing their bioavailability and thereby interfering with their apoptosis-inducing activity, provides new methods and agents for the treatment of conditions in which apoptosis of cells is desired. The apoptosis-inducing proteins which are inhibited by binding to onconeural antigens may act directly in the induction of apoptosis, such as apoptosis-inducing transcription factors, for example, those in the Myc family of oncoproteins, or such proteins may participate indirectly in induction of apoptosis by interacting with other factors to mediate apoptosis. By antagonizing the binding of onconeural antigens to the apoptosis-promoting proteins within the cell, the cells may be induced to undergo apoptosis and die. Cells in which the induction of apoptosis is desired are numerous and include both diseased and normal cells, as will be more fully elucidated below. One non-limiting example comprises dysproliferative cells such as neoplastic or cancerous cells. Such cells appear to avoid apoptosis that would occur as a result of the presence of apoptosis-inducing factors present within the cell by expressing high levels of onconeural antigens, e.g., cdr2, which bind and reduce the bioavailability of the apoptosis-inducing proteins, such as Myc. A high percentage of gynecological cancers express onconeural antigens: it has been found that cdr2 is expressed in 60% of patients with ovarian cancer, and 25% with breast cancer. Agents capable of interfering with this interaction between the onconeural antigen and the transcription factor therefor increase the bioavailability of the transcription factor, which then induces gene activation and apoptosis in dysproliferative cells.

Other uses of such agents and methods of the present invention include inducing apoptosis in normal cells; one non-limiting example is germ cells. For example, induction of apoptosis by agents or methods of the present invention of male germ cells results in the induction of sterility. This treatment provides a new method of contraception.

The invention herein is based upon several experimental observations that ascribe a role of onconeural antigens in normal cells such as neurons as preventing apoptosis by hindering nuclear entry of transcription factors. As described in the Examples below, the onconeural antigen cdr2 has been found to specifically bind to the transcription factors C-Myc and N-Myc, both known to be apoptosis-promoting proteins. Furthermore, in transcription assays dependent upon the bioavailability of Myc, cdr2 has been shown to interfere with gene expression in a dose-dependent manner, thus ascribing a role of cdr2 in binding to and blocking the activity of Myc. In such assay systems, antibodies to cdr2 increase the bioavailability of Myc and promote increased gene expression. Thus, antagonizing the onconeural antigen is a means to achieve increased transcription factor activity, and as a consequence, apoptosis.

As described in an example below, mice which are genetically deficient in the onconeural antigen Nova (Nova-1 null mice, or Nova-1 knockout mice) exhibit early apoptosis of particular motor neurons in the brain. These data in combination with the experiments described above further support the role of onconeural antigens in preventing apoptosis in normal cells by controlling the bioavailability of cell cycle-activating proteins, acting either directly or indirectly, which are present or expressed in the cell. As described above, cell cycle gene activation in certain cells, such as neurons, leads to an abortive attempt to enter the cell cycle, which results in apoptosis. Cells already in a dysregulated cell cycle, such as cancer cells, are induced to become apoptotic by such proteins. Thus, a role of onconeural antigents in tumor cells appears to be the prevention of apoptosis. A principal object of the present invention is directed toward interfering with the interaction of oncogenic antigens and their transcription factor binding partners as targets for pharmacological intervention in inducing apoptosis.

In one preferred embodiment, the present invention is directed to agents and methods for the treatment of dysproliferative diseases such as cancer, and particularly to gynecological cancers such as ovarian and breast cancer, by administration of an agent capable of antagonizing the binding of oncogenic antigens in dysproliferative cells with apoptosis-inducing proteins, thereby permitting the proteins to directly or indirectly activate cell cycle genes and thereby induce apoptosis. A non-limiting example of suitable agents for the invention include antibodies which bind to oncogenic antigens and antagonize their binding to transcription factors, fragments of antibodies which bind to oncogenic antigens, HLZ region-binding agents and others which antagonize the binding of onconeural antigens to transcription factors.

The present invention is directed the treatment of tumors, both solid and non-solid tumors. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Gynecological tumors are preferred, for example, ovarian and breast cancer. Examples of non-solid tumors include but are not limited to acute and chronic leukemias, including lymphoblastic and myeloid; lymphomas, such as Hodgkin disease, non-Hodgkin lymphoma, Burkitt lymphoma; and myelomas, such as multiple myeloma.

In another embodiment, dysproliferative changes (such as metaplasias and dysplasias) are treated or prevented in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelium; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

The present invention is also directed to treatment of non-malignant tumors and other disorders involving inappropriate cell or tissue growth by administering a therapeutically effective amount of an agent of the invention. For example, it is contemplated that the invention is useful for the treatment of arteriovenous (AV) malformations, particularly in intracranial sites. The invention may also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperproliferative disorders is also contemplated.

In another embodiment of the present invention, the agents and methods described herein may be used in the induction of apoptosis in normal cells. One tissue in which the onconeural antigen cdr2 is expressed is the testis. Induction of apoptosis in such cells would render the individual sterile. Thus, one embodiment of the present invention is in the induction of sterility, as a means of contraception. Other normal cells and tissues in which onconeural antigens are expressed and participate in the prevention of apoptosis are likewise targets of the agents and methods of the present invention for inducing apoptosis in such cells and tissues.

Methods for identifying agents useful for the practice of the present invention are apparent from the disclosure herein. Methods are disclosed for identifying agents capable of antagonizing the interaction between an onconeural antigen and an apoptosis-promoting transcription factor, such that the antagonism increases the bioavailability of the transcription factor. Such methods may be carried out in a cell-free system, in which the interactions are measured, for example, by protein detection means; or in cell-based systems, in which the interactions are detected immunohistochemically, by subcellular fractionation, or by measuring downstream effects of the interaction are detected, such as gene activation, and cellular structural or physiologic changes including cell death. The method generally is based upon the sequential steps of: (1) preparing a mixture in a cell-free or cell-based test system comprising an onconeural antigen or a fragment thereof and an apoptosis-inducing transcription factor or a fragment thereof; (2) contacting said mixture with an agent being evaluated for its ability to antagonize the interaction between said onconeural antigen and said apoptosis-inducing transcription factor; (3) evaluating the extent of interference by said agent of the interaction between said onconeural antigen and said apoptosis-inducing transcription factor; and (4) determining from said extent of interference the capability of said agent to interfere with said interaction. Agents which interfere with the interaction between the onconeural antigen and the transcription factor are suitable as agents for the methods of the present invention. The test agents may be delivered to the cell-free or cell-based mixture to initiate the test; furthermore, the effectiveness of a cellular delivery system for therapeutic effectiveness of agents of the present invention may be evaluated in a cell-based system as decribed herein.

Several methods known to one of ordinary skill in the art may be used to evaluate antagonists of the binding of onconeural antigens with transcription factors, whether by determining the extent of direct interaction between the two proteins, or their downstream effects, for example, on gene activation. Numerous in-vitro, cell-free methods are available for such assessments; non-limiting examples of such assessments are provided in the Examples here. Furthermore, in-vitro methods using whole cells are also useable, wherein the degree of interaction between the two binding partners described herein may be assessed by any one of several means, including immunohistochemical localization, by cell death, or by subcellular fractionation and quantitation of the binding partners or their upstream or downstream factors.

Non-limiting examples of suitable procedures include means for assessing directly the extent of binding of the onconeural antigen with the transcription factor by electrophoretic methods, and coprecipitation with antibody, including a pull-down assay format. In one example of the pull-down assay, a fusion protein comprising the enzyme glutathione S-transferase fused to cdr2 or a fragment of cdr2 is prepared by recombinant means. This fusion protein has the property of binding to a matrix containing immobilized glutathione. An agent suspected of interacting with cdr2 may be added to the fusion protein immobilized to matrix, after which labeled transcription factor, such as ³⁵S-myc may be added, and the degree of binding of the labeled myc determined by any one of a number of procedures, including SDS-PAGE and fluorography, direct determination of label bound to the matrix, and others.

In a further example of an assay for assessing whether agents interfere with the binding of onconeural antigens with transcription factors, the gene activation capability of the transcription factor can be assessed. In the absence of an agent which interferes with the binding of the onconeural antigen with the transcription factor, no gene activation will occur. Antagonists of the binding will make available free transcription factor. In an example of such an assay, cells transfected with a reporter plasmid are used in which the bioavailability of a transcription factor will result in the expression of the reporter gene. The cells may be co-transfected with a plasmid expressing an onconeural antigen such as cdr2, and further with a plasmid expressing a transcription factor such as myc. Exposure of the cell to agents which antagonize the interaction between the onconeural antigen and the transcription factor results in increased expression of the reported gene.

In a further example of a method to assess the antagonism of the binding of an onconeural antigen to a transcription factor, immunohistochemical or subcellular fractionation methods may be used to localize the transcription factor. Cells may be selected or engineered to optimize the sensitivity of the assay. Under normal conditions in which no antagonist of the interaction is present, a quantifiable amount of the transcription factor will be held in the cytoplasm by association with the onconeural antigen a corresponding amount of the factor will be detectable in the nucleus. In the presence of an antagonist, increased nuclear localization of the transcription factor will occur, which may be quantified by the above-described techniques, among others.

In a further embodiment, the assessment of the activity of compounds to induce apoptosis may be measured in a cell-based assay system where cell death is the indicator of activity.

By means of the aforementioned methods for identifying suitable agents of the present invention, agents suitable for the practice of the present invention comprise those capable of antagonizing the interaction between an onconeural antigen and an apoptosis-promoting protein. One non-limiting example of the type of agents that are suitable for the practice of the present invention are antibodies to the onconeural antigen, which interfere with the association between the onconeural antigen and the apoptosis-inducing protein. Examples wherein the interaction is disrupted using antibodies to onconeural antigens is shown in the non-limiting examples below. Included in this group of agents are antibody fragments and recombinant constructs with the antigen-binding domain, among other modified molecules containing the antigen-binding region. As a non-limiting example of another type of agent capable of interfering with the interaction, onconeural antigens such as cdr2 possess, as described above, an helix-leucine zipper (HLZ) region which is the site of interaction with the HLZ region on transcription factors such as Myc. Agents capable of interacting with the HLZ region of the transcription factor and competing with or interfering with the binding of the onconeural antigen will achieve the object of the invention in increasing the bioavailability of free transcription factor and allowing the transcription factor to enter the nucleus and induce gene activation. As an example of this embodiment of the invention, an HLZ-containing fragment of an onconeural antigen such as fragment of cdr2 comprising amino acids 16 to 192 (SEQ ID NO.:1) or amino acids 65-140 (SEQ ID NO:2), are suitable HLZ-containing fragments known to interact and bind to the transcription factors such as C-Myc and N-Myc. Studies described herein demonstrate binding of c-Myc to said 16-192 amino acid fragment of cdr2 coupled to GST in a fusion protein. Other HLZ region-containing fragments are intended as agents of the present invention. The present invention is not intended to be limited to a particular apoptosis-inducing factor or structural feature thereof.

With regard to one embodiment wherein agents of the present invention comprise antibodies, such agents may be selected from polyclonal, monoclonal, chimeric, single chain, Fab fragments, and recombinant constructs comprising the antigen-binding domain.

In accordance with the present invention, onconeural antigens isolated from tissues or produced recombinantly or by chemical synthesis, and fragments or other derivatives or analogs thereof, including fusion proteins, may be used as an immunogen to generate antibodies that recognize the onconeural antigen. Such antibodies include but are not limited to polyclonal, monoclonal, chimeric, single chain, Fab fragments, and an Fab expression library. Preferably, such an antibody is specific for a human onconeural antigen.

Various procedures well known in the art may be used for the production of polyclonal antibodies to onconeural antigens or derivatives or analogs thereof. For the production of antibody, various host animals can be immunized by injection with the immunogen described above. In one embodiment, the oncogenic antigen or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the onconeural antigen or fragment analog, or derivative thereof, any technique that provides for the production of antibody molecules by continuous cell lines in culture may be used. Human monoclonal antibodies are preferred. These include but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. In an additional embodiment of the invention, monoclonal antibodies can be produced in germ-free animals utilizing recent technology [PCT/US90/02545]. In fact, according to the invention, techniques developed for the production of “chimeric antibodies” [Morrison et al., J. Bacteriol. 159:870 (1984); Neuberger et al., Nature 312:604-608 (1984); Takeda et al., Nature 314:452-454 (1985)] by splicing the genes from a mouse antibody molecule specific for an onconeural antigen together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce onconeural antigen-specific single chain antibodies. An additional embodiment of the invention utilizes the techniques described for the construction of Fab expression libraries [Huse et al. Science 246:1275-1281 (1989)] to allow rapid and easy identification of monoclonal Fab fragments with the desired specificity for an onconeural antigen, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the second embodiment of the agents of the present invention, peptides and polypeptides which comprise an HLZ-interacting region are suitable for the practice of the present invention. Such peptides or polypeptides may be prepared by chemical synthetic or recombinant means. Such methods for the preparation of peptides and proteins are well known to one of ordinary skill in the art.

The methods used herein may be used to identify other molecules capable of achieving the desired objective of the agents of the present invention in antagonizing the interaction between an onconeural antigen and an apoptosis-inducing transcription factor.

In yet another aspect of the present invention, provided are pharmaceutical compositions of the above-described agents. Such pharmaceutical compositions may be for administration for injection, or for oral, pulmonary, nasal or other forms of administration. In general, comprehended by the invention are pharmaceutical compositions comprising effective amounts of a low molecular weight component or components, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form.

In yet another aspect of the present invention, methods of treatment and manufacture of a medicament are provided. Conditions alleviated or modulated by the administration of the present derivatives are those indicated above.

For all of the above molecules, as further studies are conducted, information will emerge regarding appropriate dosage levels for treatment of various conditions in various patients, and the ordinary skilled worker, considering the therapeutic context, age and general health of the recipient, will be able to ascertain proper dosing. Generally, for intravenous injection or infusion, or with a derivative of the component, dosage may be lower. The dosing schedule may vary, depending on the circulation half-life, and the formulation used.

According to the invention, the component or components of a therapeutic composition of the invention may be introduced parenterally, transmucosally, e.g., orally, nasally, or rectally, or transdermally. Preferably, administration is parenteral, e.g., via intravenous injection, and also including, but is not limited to, intraarterial, intramuscular, intradermal, subcutaneous, intraperitoneal, intraventricular, and intracranial administration. More preferably, where administration of the onconeural antagonist is indicated to induce apoptosis of a tumor, it may be introduced by injection into the tumor or into tissues surrounding the tumor.

In another embodiment, the therapeutic compound can be delivered in a vesicle, in particular a liposome [see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.]. To reduce its systemic side effects, this may be a preferred method for administration. Furthermore, means of administration known to facilitate the uptake of the particular type of agent of the present invention into tumor cells is preferred.

In a further embodiment, in the example of the treatment of dysproliferative diseases such as cancer, it is desirable to administer the agent of the present invention to the dysproliferative cells of a patient and minimize the effects of the agent on non-dysproliferative cells, especially cells wherein the interaction between the onconeural antigen and the transcription factor is maintaining homeostasis. In particular, avoidance of delivery of the agents of the present invention to the central nervous system is desired. Since cdr2 is expressed in tumor cells, neurons, and testis (Matsui, 1998), by reducing lipid solubility, an agent of the present invention may have limited ability to cross the blood-brain barrier, thus providing an increased therapeutic index for the treatment of tumors and avoiding effects on neurons. cdr2 is also expressed in testis, a factor not relevant for the treatment of gynecological tumors. In another embodiment of the invention, cells of the body may be exposed to the agent of the present invention ex vivo, and returned to the body after exposure and, optionally, washing, to deliver the agent to circulating dysproliferative cells.

As is shown herein in the Examples below, the paraneoplastic cerebellar degeneration target antigen cdr2 binds to Myc in vivo and down regulates Myc function in co-transfection assays. These findings may be extended to indicate that cytoplasmic cdr2 acts to down regulate Myc function as a nuclear transcription factor.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### cdr2 Binds Selectively to Myc

A yeast 2-hybrid screen was used to identify binding between cdr2 and Myc. The amino-terminal 150 amino acids of cdr2 contain an acidic region of 30 amino acids, followed by an extended amphipathic helix of 100 amino acids and a classical leucine zipper dimerization motif. Several N-terminal constructs were tested for activation in a yeast two-hybrid system, as follows. A pJG4-5-based HeLa cDNA library encoding B42 activation domain fusion proteins under control of the Gal1 promoter was kindly provided by Dr. Roger Brent. The host strain for all assays was EGY48 (MATa trp1 ura3 his3 LEU2::pLexAop6-LEU2), in which the endogenous LEU2 gene has been replaced by a LEU2 reporter harboring six LexA binding sites. The cdr2⁶⁵⁻¹⁴⁰ bait was tested for its ability to activate the LEU2 reporter gene independently and to enter the nucleus prior to library screening. Specificity of the yeast two-hybrid interaction was tested in yeast by the amount of growth on Leu-media and β-galactosidase (β-gal) expression. Significant growth and β-gal expression were evident when Myc was present with the LexA/cdr2⁶⁵⁻¹⁴⁰ bait construct in the presence of galactose but not glucose. Similarly, there was no interaction of Myc with a Drosophila bicoid bait construct. Conversely, cdr2⁶⁵⁻¹⁴⁰ interacted with Myc but not Max or Mxi1 constructs (data not shown). pJG4-5 plasmids encoding Max and Mxi1 activation domain fusion proteins were kindly provided by Dr. Erica Golemis.

It was found that constructs containing the cdr2 HLZ domain without the acidic domain were suitable for screening. Myc was identified as a specifically interacting clone, as shown in Table 1.

TABLE 1

| Bait | GAL4-cDNA | lacZ | leu- |
|---|---|---|---|
| cdr2 | c-myc | +++ | +++ |
| bicoid | c-myc | − | − |
| cdr2 | max | − | not determined |
| cdr2 | mxil | − | not determined |

As shown in Table 1, cdr2 bound strongly to Myc, but did not bind constructs expressing Max or bicoid. Thus, cdr2 binds specifically to Myc in the yeast two-hybrid system.

### EXAMPLE 2

### Confirmation of the Selectivity of Binding of cdr2 to myc

To confirm these findings, and to demonstrate that the interaction between cdr2 and Myc is not dependent on additional yeast proteins, protein interactions were directly assayed in vitro using GST fusion proteins and ³⁵S-labeled in vitro translation products, in the form of a pull-down assay. Plasmids encoding GST fusion with Max, USF, Myc353 (Myc amino acids 250-353) and Myc439 (Myc amino acids 250-439) and a plasmid containing the full length cDNA of mouse Myc were kindly provided by Dr. K. Calame (Shrivastava, et al., 1993). Full length mouse Myc and mouse cdr2 RNAs were transcribed in vitro using T7 RNA polymerase (Promega), and those RNAs were translated in vitro using rabbit reticulocyte lysate system (Promega) with ³⁵S-L-methionine (Amersham). All fusion proteins were expressed in bacteria and affinity purified with glutathione sepharose (Pharmacia). The GST-cdr2 fusion protein containing amino acids 16-192 of human cdr2 was also purified using glutathione sepharose affinity chromatography. In vitro binding assays were performed essentially as described (Harper et al., 1993). Briefly, GST fusion proteins were immobilized to glutathione sepharose and washed with binding buffer (50 mM Tris HCl, pH 7.5, 120 mM NaCl, 2 mM EDTA, 0.1% Nonidet P-40, 1 mM NaF, 2 μg/ml aprotinin, 100 μg/ml PMSF). Labeled proteins were incubated with immobilized fusion proteins and after washing unbound proteins, the samples were separated by SDS-PAGE and analyzed by fluorography.

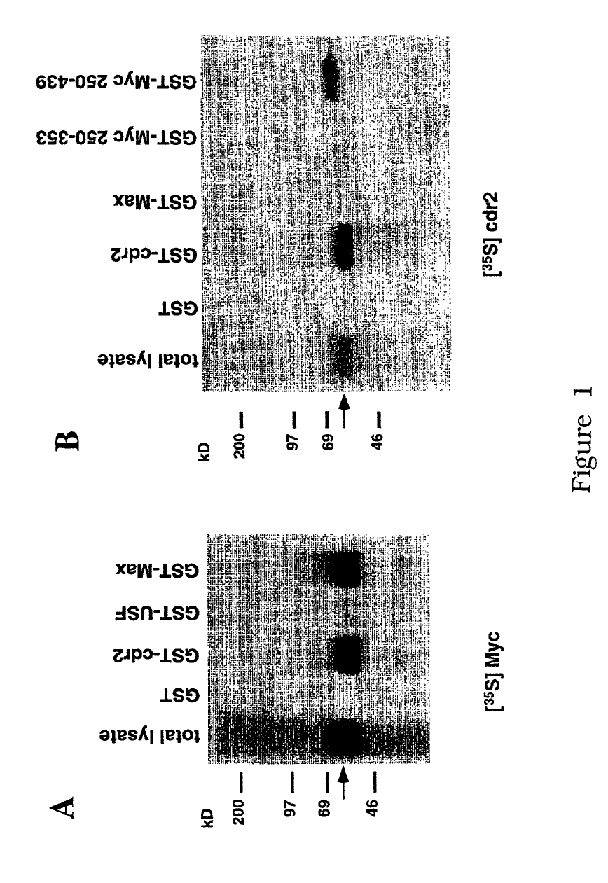

³⁵S-labeled Myc showed equally robust interactions with Max and cdr2, but failed to react with control proteins (FIG. 1A). Conversely, ³⁵S-labeled cdr2 reacted with Myc and cdr2 itself suggesting that the protein may be able to homodimerize through its leucine zipper domain (FIG. 1B). Cdr2 failed to interact with Max or with Myc deletion constructs lacking the C-terminal HLZ domain (FIG. 1B). These results demonstrate a direct and specific interaction between cdr2 and the Myc HLZ domain.

### EXAMPLE 3

### Co-Localization of cdr2 and Myc in Cerebellar Purkinje Neurons

To determine whether adult cerebellar Purkinje neurons express Myc and to assess where the protein is localized, rat brain sections were examined by immunohistochemistry using a Myc monoclonal antibody, and compared with the staining obtained with cdr2 antibody.

Rat brains perfused with 4% paraformaldehyde/PBS were post-fixed in 4% paraformaldehyde/PBS at 4° C. for 4 hours and stored in 10% sucrose/PBS overnight. Floating sections (30 μm) were blocked in PBS/0.05% triton X-100/2% normal horse serum (blocking buffer). c-Myc monoclonal antibody C-33 (Santa Cruz Biotechnology) diluted in blocking buffer was used at 1 μg/ml, and PND antisera was used at 1:200 dilution. After washing with PBS/0.05% triton X-100, sections were incubated with biotinylated secondary antibodies (Vector Laboratories) and washed. Signals were enhanced by addition of HRP-conjugated avidin (Vector Laboratories), developed with diaminobenzidene (DAB) in the presence of H2O2, and visualized by light microscopy using a Zeiss Axioplan microscope.

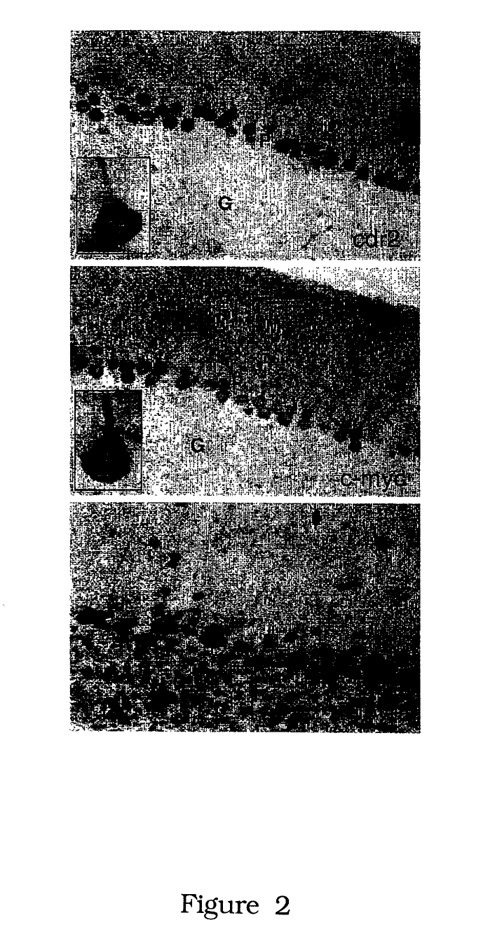

FIG. 2 demonstrates that Myc and cdr2 show a striking co-localization in the cytoplasm of Purkinje neurons. As a positive control for nuclear reactivity in these fixation conditions, we stained a serial section for the Nova protein, which is abundantly expressed in the Purkinje cell nucleus (FIG. 2C); in addition, Myc could also be detected in the nuclei of some Purkinje neurons (data not shown). The overlap in cdr2 and Myc localization is consistent with a direct association of the proteins in vivo.

### EXAMPLE 4

### Identification of cdr2-Myc Interactions Using Co-Immunoprecipitation

To examine whether cdr2 and Myc interact in vivo, coimmunoprecipitation experiments were performed using mouse cerebellum. Mouse cerebellum was homogenized in LS lysis buffer (20 mM HEPES, pH 7.5, 100 mM KCl, 10 mM MgCl2, 5 mM dithiothreitol (DTT), 0.2% NP-40, 1, 2μg/ml aprotinin, 0.2 mM phenylmethylsulfonyl fluoride (PMSF)) using a dounce homogenizer. Homogenates were sonicated briefly and soluble fractions were collected after spinning. The lysates were precleared with Protein A-sepharose and normal rabbit sera, and then precipitated with PCD CSF or anti-Myc rabbit polyclonal antibody (Upstate Biotechnology). Immunoprecipitated proteins were separated by SDS-PAGE and transferred to nitrocellulose filters. Blotted proteins were analyzed using anti-cdr2 patients' sera or anti-Myc mouse monoclonal antibody (C-33, Santa Cruz Biotechnology). Each protein was visualized in the blot using HRP-conjugated anti-human or anti-mouse IgG and ECL chemiluminescence kit (Amersham).

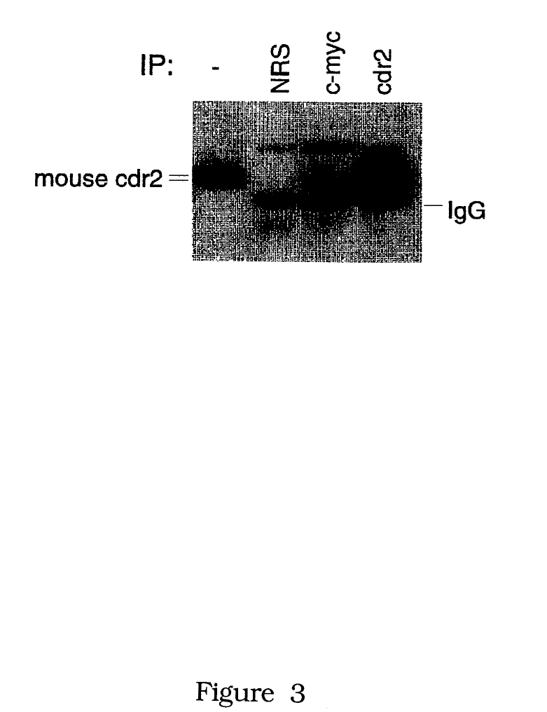

Myc antibodies were able to precipitate Myc itself, and to coimmunoprecipitate cdr2 from solubilized whole cerebellar extracts, demonstrating the existence of a cdr2:Myc complex (FIG. 3). In these SDS-PAGE gels, which were run under highly resolving conditions to separate cdr2 from IgG, cdr2 was detected as a doublet of ˜55 kD (FIG. 3). Interestingly, Myc appeared to preferentially immunoprecipitate the smaller of the two cdr2 bands, suggesting that Myc may specifically interact with a unique cdr2 species. Neither of these cdr2 reactive proteins were immunoprecipitated by non-specific rabbit immunoglobulin (FIG. 3). Preincubation of Myc antibodies with immunogenic peptide abolished both Myc immunoprecipitation and cdr2 coimmunoprecipitation (data not shown). Notably, while cdr2 antibody was able to precipitate cdr2 protein from cerebellar extracts, it failed to coimmunoprecipitate Myc protein under a variety of conditions, although coimmunoprecipitation of T7-tagged cdr2 with Myc in transfected cells was achieved using a T7 monoclonal antibody (see below).

### EXAMPLE 5

### Identification of cdr2-N-Myc Interactions Using Co-Immunoprecipitation

To examine whether cdr2 and N-Myc interact in vivo, coimmunoprecipitation experiments were performed using mouse cerebellum. The same procedure was followed as described in Example 4, except that anti-Myc rabbit polyclonal antibody [source??] was used. Mouse cerebellar homogenate was run on Western blot before (lane 1) of after immunoprecipitation with the indicated antibodies: anti-cdr2 PCD patient's cerebrospinal fluid (CSF) (lane 2); normal rabbit antiserum (lane 3); and anti-N-Myc rabbit polyclonal antibody (lane 4). Immune complexes were analyzed by Western blot using anti-cdr2 patient's antiserum. The arrow points to the cdr2 protein band.

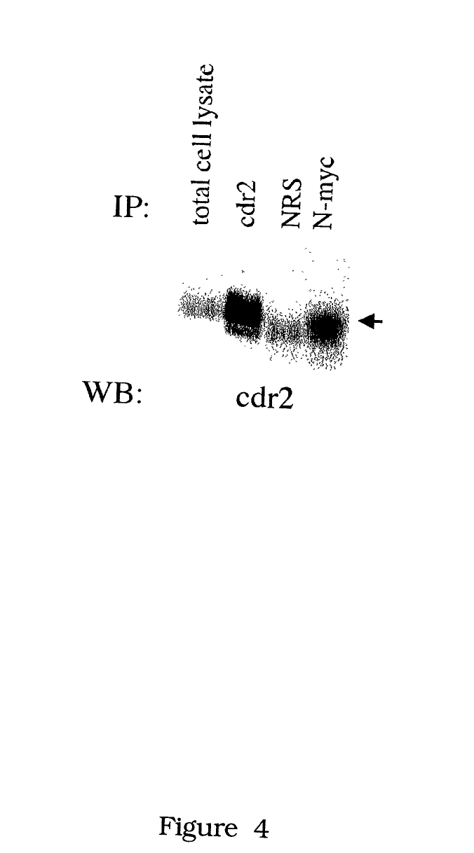

Anti-N-Myc antibodies were able to precipitate N-Myc itself, and to coimmunoprecipitate cdr2 from solubilized whole cerebellar extracts, demonstrating the existence of a cdr2:N-Myc complex (FIG. 4).

### EXAMPLE 6

### The Effect of the cdr2:Myc Interaction on Gene Expression

To determine whether the cdr2:Myc interaction could alter the ability of Myc to induce gene expression, Myc-induced transcription of a reporter construct in the presence or absence of cdr2 was examined. Rat 1A fibroblast cell lines were transiently transfected with a reporter gene which either had (M4 minCAT) or did not have (minCAT) Myc binding sites (E-box elements) upstream of a CAT reporter gene, as follows. The pSpMyc and (+/−)M4 minCAT plasmids used in transfection assays were kindly provided by Dr. R. Eisenman. For CAT assays, transfected cells were lysed in 0.25M Tris HCl, pH 7.5 by repeated freezing and thawing. Cytoplasmic extracts were mixed with CAT assay buffer (2 μCi/ml 14C-chloramphenicol, 0.25 mg/ml n-butyryl CoA, 16.6 mM Tris HCl, pH 8.0), and incubated at 37° C. To isolate the acetylated ¹⁴C-chloramphenicol, samples were extracted by 2:1 mixture of tetramethyl pentadecane (TMPD)/xylene. Radioactivity in the extracted organic phase was measured using a liquid scintillation counter. Luciferase assays were done using a luciferase assay kit (Promega) as described by the manufacturer. Transfection efficiency was normalized by measuring the β-gal activity derived from cotransfection with a CMV-lacZ reporter construct, and, in some instances normalizing the number of cells transfected using a pEGFP reporter (Clontech). To measure the β-gal activity, cytoplasmic extracts were mixed with Buffer A (100 mM NaH₂PO₄, pH 7.5, 10 mM KCl, 1 mM MgSO₄, 50 mM 2-mercaptoethanol) and 4 mg/ml O-nitrophenyl D-β-galactopyranoside (ONPG). After incubation, reactions were stopped by adding 1 M Na2CO3 and the absorbance at 420 nm was measured.

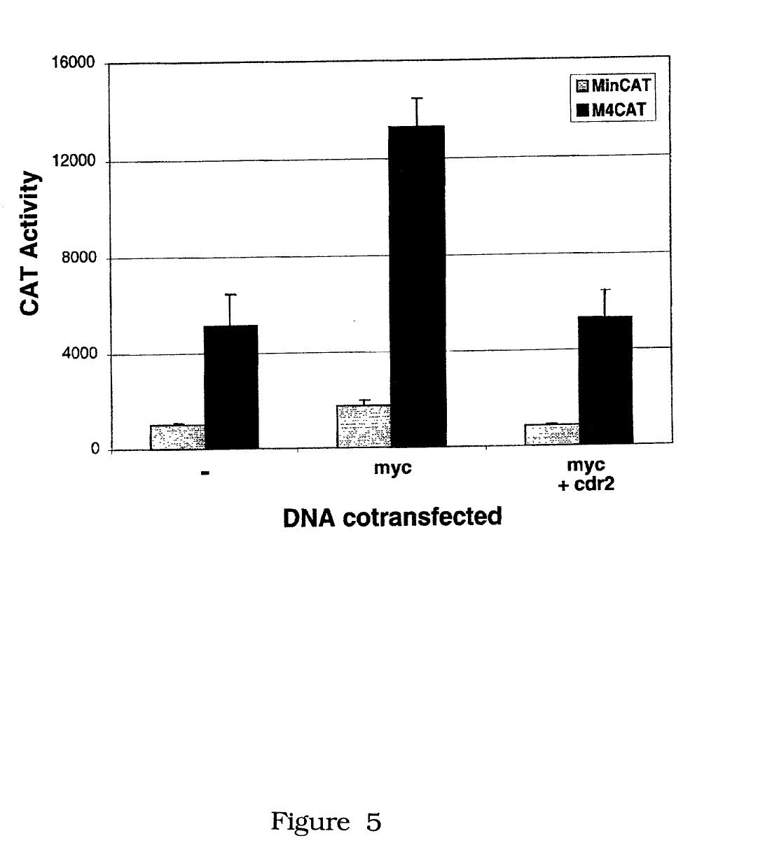

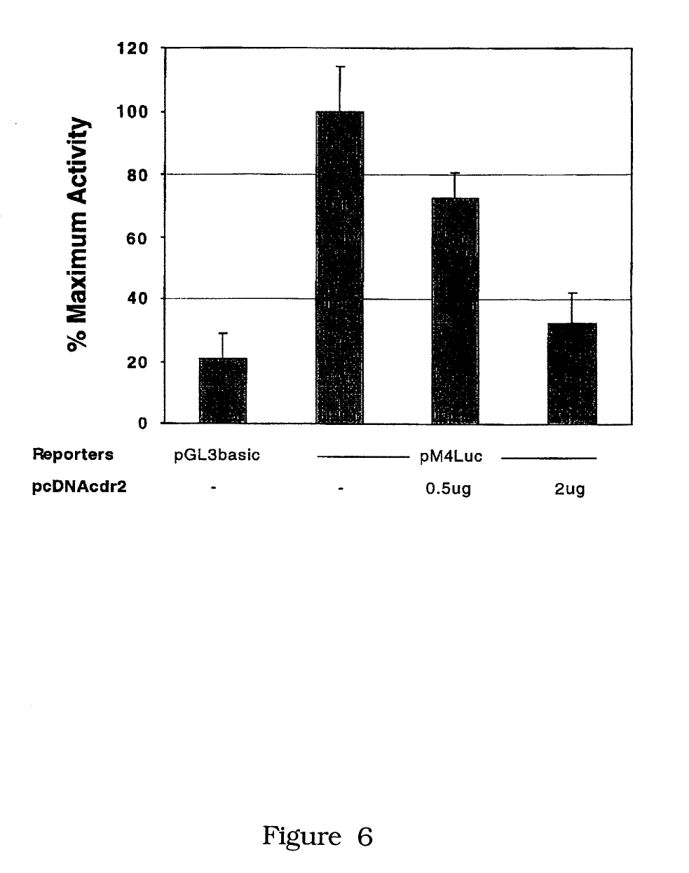

In the absence of transfected Myc, baseline transcription from endogenous cellular Myc proteins leads to specific CAT induction from the M4 minCAT construct (FIG. 5, “−” lane; Kretzner et al., 1992). Transfection of a Myc expressing plasmid led to a 2.6 fold increase in E-box dependent CAT activity, which was suppressed by co-transfection with cdr2 (FIG. 5). To confirm and extend these observations, we examined whether titrating increasing amounts of cdr2 yielded a dose-dependent effect on Myc dependent transcription. For these experiments, we assayed transcription using a luciferase reporter construct in NIH-3T3 cells transfected with a myc expressing plasmid. We found that a luciferase reporter harboring E-box elements (pM4luc) showed approximately 5 fold more activity than a basal promoter construct (FIG. 6, lanes 1-2). Co-transfection of increasing concentrations of cdr2 together with the pM4luc reporter led to a linear decrease in Myc-dependent transcriptional activity, over a four-fold range (FIG. 6). These results indicate that cdr2 was able to block the action of co-transfected Myc to induce E-box dependent transcription.

### EXAMPLE 7

### cdr2 Induces Redistribution of Myc in the Cytoplasm

To further evaluate a possible functional role for the interaction of cdr2 and Myc, cdr2 was stably expressed under the control of a tetracycline inducible promoter in HTC-75 cells. Tet-inducible cdr2 expression HTC-75 cells and the HT1080-derived tet-suppressive cell line were provided by Dr. T. de Lange (van Steensel and de Lange, 1997). Those cells were kept in DME media supplemented with 10% fetal bovine serum and antibiotics. Full length mouse cdr2 cDNA linked to the T7tag sequence was cloned into pUHD10-3 plasmid. DNA was transferred to HTC-75 cells using Lipofectamine (GIBCO-BRL) as described by the manufacturer. Stable transformants were selected and cloned by adding Geneticin (GIBCO-BRL) to 400 μg/ml. The expression level of each clone was examined by western blot after removing doxycyclin from media.

HTC-75 cells were incubated for 72 hrs in the presence (control; cdr2 expression off) or absence (cdr2; cdr2 expression on) of doxycyclin. Fixed cells were stained with affinity purified cdr2 antibodies (green) and Myc monoclonal antibodies (red) and examined by confocal microscopy (Zeiss). Colocalization was examined by a combination of red and green fluorescence (yellow).

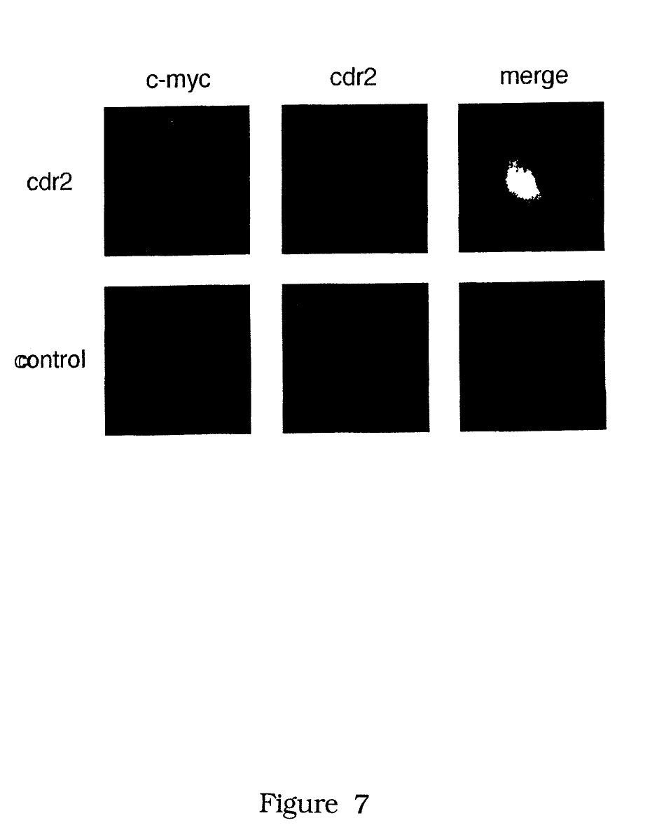

In the absence of induction of the cdr2 expression construct, no detectable cdr2 protein could be detected, while Myc showed diffuse cytoplasmic and nuclear staining (FIG. 7). Upon induction of cdr2 expression, cdr2 protein was present in a perinuclear cluster in the cytoplasm, although some protein could be detected diffusely in the cytoplasm; no protein could be detected within the nucleus. Under these conditions, the cytoplasmic Myc showed a striking redistribution, colocalizing with cdr2 to the perinuclear clusters.

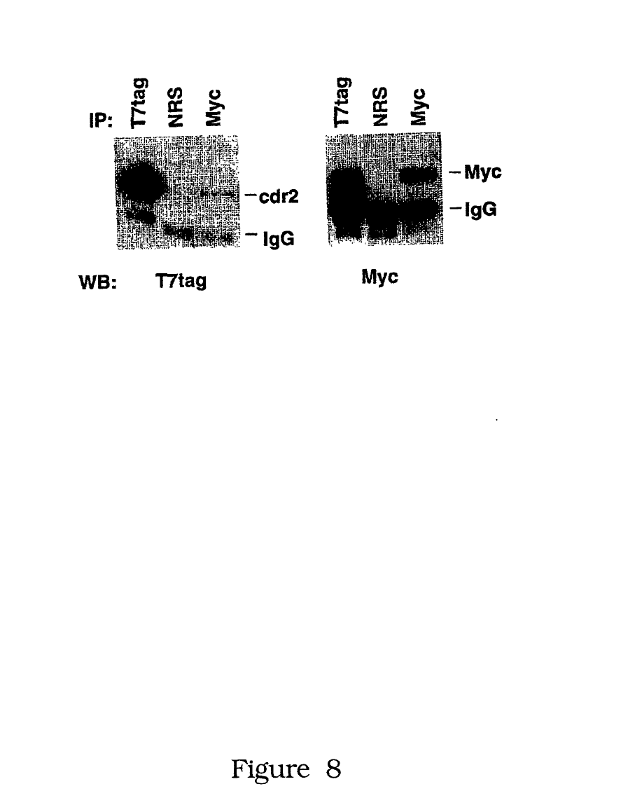

To evaluate whether cdr2 and Myc directly interact in these cells, coimmunoprecipitation experiments were performed, using either Myc antibody or a T7 antibody specific to an epitope tag encoded in the cdr2 expression construct. As demonstrated with cerebellar extracts, Myc antibody was able to precipitate Myc protein and coprecipitate cdr2 protein (FIG. 8). Conversely, T7 antibody was able to precipitate the T7 tagged cdr2 protein and coprecipitate Myc protein (FIG. 8). No T7 tagged cdr2 protein could be detected in nuclear fractions (data not shown). Taken together, these data demonstrate that expression of the cdr2 protein directly interacts with Myc in the cytoplasm, resulting in a change in its cytoplasmic distribution.

### EXAMPLE 8

### PCD Disease Antisera Inhibit the cdr2:Myc Interaction

The previous study suggested that PCD antisera might inhibit the interaction between cdr2 and Myc. PCD antisera were then examined to determine whether antibodies present therein could affect the interaction between cdr2 and Myc. PCD antisera have been reported to recognize an epitope in the region of the cdr2 HLZ domain (Sakai et al., 1993). The determination was performed as follows. GST-cdr2 or GST-Max fusion proteins in solution were immobilized on glutathione-sepharose beads and incubated with patients' sera. After washing, in vitro translated ³⁵S-Myc proteins were added to each tube. After washing again, proteins present on GST-cdr2 or GST-Max sepharose beads were analyzed by SDS-PAGE and fluorography.

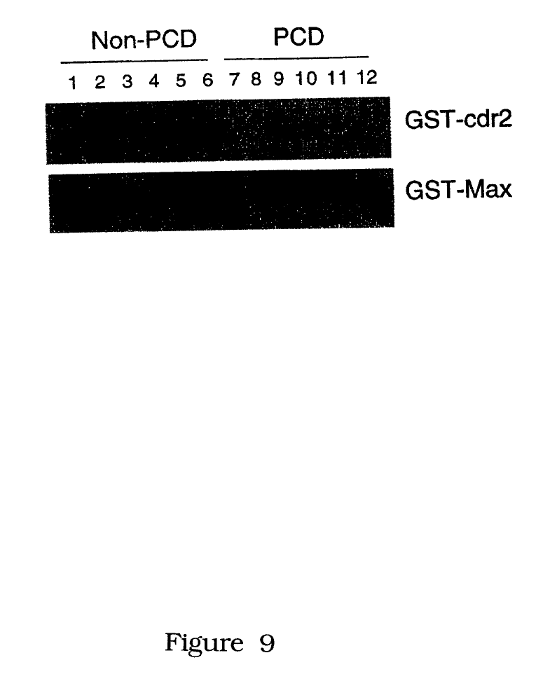

All six PCD disease antisera significantly inhibited the interaction of cdr2 with Myc (FIG. 9) relative to the non-PCD control sera. Moreover, the PCD antisera failed to affect the interaction of Max with Myc. Quantitation of these data revealed a 5.5 fold inhibition of the cdr2:Myc interaction by PCD antisera, relative to control sera. These results indicate that a hallmark of PCD disease antisera is an ability to disrupt the interaction of cdr2 with Myc in vitro.

### EXAMPLE 9

### Histopathology of Nova-1 Null Mice

Mice lacking the onconeural antigen Nova gene, termed Nova-1 null mice, were prepared by blastocyst injection of embryonic stem (ES) cells. These ES cells harbor a homologous recombination such that the initiating methionine in the Nova-1 gene is deleted (null mutation). No Nova-1 protein is produced in mice homozygous for this null mutation.

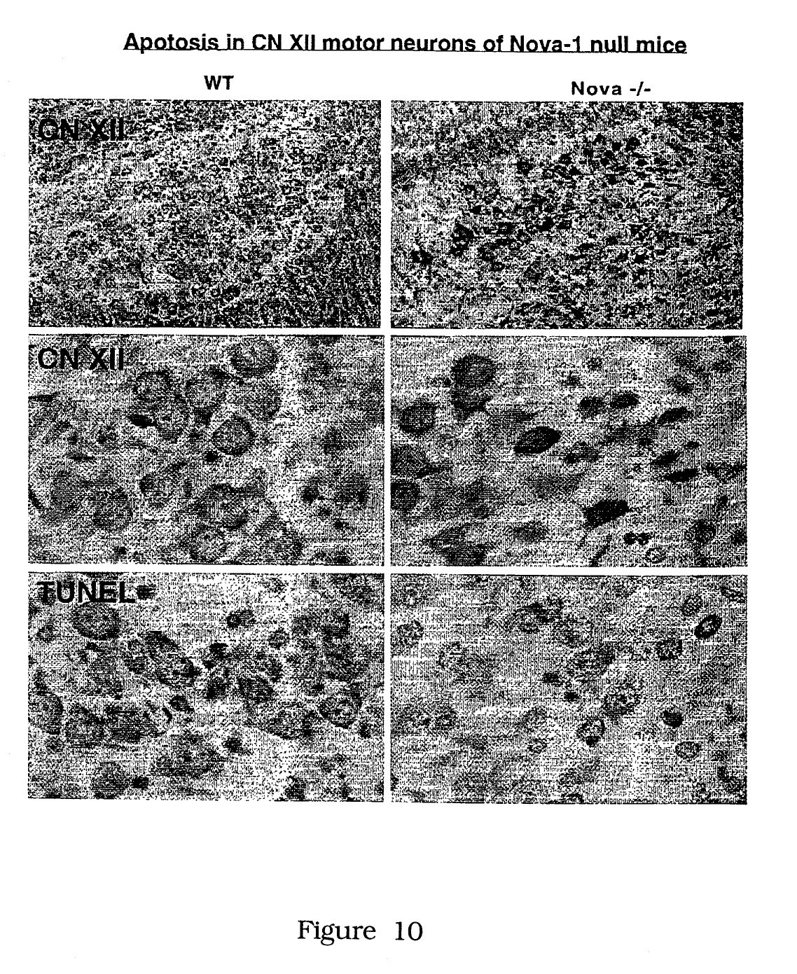

Histopathological examination of CN XII motor neurons in Nova-1 null (knockout) mice, as compared to wild-type mice (FIG. 10), shows selective degeneration of motor neurons in the hypoglossal nucleus of the Nova-1 null mice. The most severe pathology has been found in much that have been found shortly after death.

Pathologic neurons show pyknotic nuclei typical of neurons undergoing apoptosis. Specific evidence for apoptosis is shown in the panels marked TUNEL, a study of mice sacrificed while relatively healthy at postnatal day zero. In this preliminary study, the nuclei of large motor neurons in CN XII of wild type animals show only background staining, while discrete nuclear staining is evident in the motor neurons in CN XII of Nova-1 null mice.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

Albert, M. L., Darnell, J. C., Bender, A., Francisco, L., Bhardwaj, N. and Darnell, R. B. (1998). Tumor-specific killer cells in paraneoplastic cerebellar degeneration. Nature Med in press.

Ayer, D. E., Kretzner, L. and Eisenman, R. N. (1993). Mad: a heterodimeric partner for Max that antagonizes Myc transcriptional activity. Cell 72: 211-22.

Bai, M. K., Costopoulos, J. S., Christoforidou, B. P. and Papadimitriou, C. S. (1994). Immunohistochemical detection of the c-myc oncogene product in normal, hyperplastic and carcinomatous endometrium. Oncology 51: 314-9.

Blackwood, E. M., Luscher, B. and Eisenman, R. N. (1992). Myc and Max associate in vivo. Genes Dev 6: 71-80.

Boni, R., Bantschapp, O., Muller, B. and Burg, G. (1998). c-myc is not useful as prognostic immunohistochemical marker in cutaneous melanoma. Dermatology 196: 288-91.

Borges, L. F., Elliott, P. J., Gill, R., Iversen, S. D. and Iversen, L. L. (1985). Selective extraction of small and large molecules from the cerebrospinal fluid by Purkinje neurons. Science 228: 346-348.

Bouchard, C., Staller, P., Eilers, M. (1998). Control of cell proliferation by Myc. Trends in Cell Biology 8:202-206.

Corradi, J. P., Yang, C. W., Darnell, J. C., Dalmau, J. and Darnell, R. B. (1997). A post-transcriptional regulatory mechanism restricts expression of the paraneoplastic cerebellar degeneration antigen cdr2 to immune privileged tissues. J Neurosci 17: 1406-1415.

Corriveau, R. A., Huh, G. S. and Shatz, C. J. (1998). Regulation of class I MHC gene expression in the developing and mature CNS by neural activity. Neuron 21: 1-20.

Craig, R. W., Buchan, H. L., Civin, C. I. and Kastan, M. B. (1993). Altered cytoplasmic/nuclear distribution of the c-myc protein in differentiating ML-1 human myeloid leukemia cells. Cell Growth Differ 4: 349-57.

DiDonato, J. A., Hayakawa, M., Rothwarf, D. M., Zandi, E. and Karin, M. (1997). A cytokine-responsive IkappaB kinase that activates the transcription factor NF-kappaB. Nature 388: 548-54.

Dropcho, E., Chen, Y., Posner, J. and Old, L. (1987). Cloning of a brain protein identified by autoantibodies from a patient with paraneoplastic cerebellar degeneration. Proc Natl Acad Sci USA 84: 1-5.

Evan, G. and Littlewood, T. (1998). A matter of life and cell death. Science 281: 1317-21.

Fabian, R. H. and Petroff, G. (1987). Intraneuronal IgG in the central nervous system: uptake by retrograde axonal transport. Neurology 37: 1780-1784.

Fathallah-Shaykh, H., Wolf, S., Wong, E., Posner, J. and Furneaux, H. (1991). Cloning of a leucine-zipper protein recognized by the sera of patients with antibody-associated paraneoplastic cerebellar degeneration. Proc Natl Acad Sci USA 88: 3451-3454.

Feddersen, R. M., Clark, H. B., Yunis, W. S. and Orr, H. T. (1995). In vivo viability of postmitotic Purkinje neurons requires pRb family member function. Mol Cell Neurosci 6: 153-167.

Feddersen, R. M., Ehlenfeldt, R., Yunis, W. S., Clark, H. B. and Orr, H. T. (1992). Disrupted cerebellar cortical development and progressive degeneration of Purkinje cells in SV40 T antigen transgenic mice. Neuron 9: 955-966.

Flaris, N. A., Densmore, T. L., Molleston, M. C. and Hickey, W. F. (1993). Characterization of microglia and macrophages in the central nervous system of rats: definition of the differential expression of molecules using standard and novel monoclonal antibodies in normal CNS and in four models of parenchymal reaction. Glia 7: 34-40.

Furneaux, H. L., Reich, L. and Posner, J. P. (1990). Autoantibody synthesis in the central nervous system of patients with paraneoplastic syndromes. Neurology 40: 1085-1091.

Galaktionov, K., Chen, X. and Beach, D. (1996). Cdc25 cell-cycle phosphatase as a target of c-myc. Nature 382: 511-7.

Graus, F., Illa, I., Agusti, M., Ribalta, T., Cruz-Sanchez, F. and Juarez, C. (1991). Effect of intraventricular injection of an anti-Purkinje cell antibody (anti-Yo) in a guinea pig model. J Neurol Sci 106: 82-87.

Green, D. R. (11997). A Myc-induced apoptosis pathway surfaces. Science 278: 1246-7.

Groyer, A., Schweizer-Groyer, G., Cadepond, F., Mariller, M. and Baulieu, E. E. (11987). Antiglucocorticosteroid effects suggest why steroid hormone is required for receptors to bind DNA in vivo but not in vitro. Nature 328: 624-6.

Harper, J. W., Adami, G. R., Wei, N., Keyomarsi, K. and Elledge, S. J. (1993). The p21 Cdk-interacting protein Cip1 is a potent inhibitor of G1 cyclin-dependent kinases. Cell 75: 805-16.

Heintz, N. (1993). Cell death and the cell cycle: a relationship between transformation and neurodegeneration? Trends Biochem Sci 18: 157-9.

Hida, C., Tsukamoto, T., Awano, H. and Yamamoto, T. (1994). Ultrastructural localization of anti-Purkinje cell antibody-binding sites in paraneoplastic cerebellar degeneration. Arch Neurol 51: 555-8.

Hurlin, P. J., Queva, C. and Eisenman, R. N. (11997). Mnt, a novel Max-interacting protein is coexpressed with Myc in proliferating cells and mediates repression at Myc binding sites. Genes Dev 11: 44-58.

Hurlin, P. J., Queva, C., Koskinen, P. J., Steingrimsson, E., Ayer, D. E., Copeland, N. G., Jenkins, N. A. and Eisenman, R. N. (11996). Mad3 and Mad4: novel Max-interacting transcriptional repressors that suppress c-myc dependent transformation and are expressed during neural and epidermal differentiation. Embo J 15: 203.

Kretzner, L., Blackwood, E. M. and Eisenman, R. N. (11992). Myc and max proteins possess distinct transcriptional activities. Nature 359: 426-429.

Lenschow, D. J., Walunas, T. L. and Bluestone, J. A. (1996). CD28/B7 system of T cell costimulation. Annu Rev Immunol 14: 233-58.

Lipponen, P. K. (1995). Expression of c-myc protein is related to cell proliferation and expression of growth factor receptors in transitional cell bladder cancer. J Pathol 175: 203-10.

McCormack, M. A., Rosen, K. M., Villa-Komaroff, L. and Mower, G. D. (1992). Changes in immediate early gene expression during postnatal development of cat cortex and cerebellum. Brain Res Mol Brain Res 12: 215-23.

McMahon, S. B., Van Buskirk, H. A., Dugan, K. A., Copeland, T. D. and Cole, M. D. (1998). The novel ATM-related protein TRRAP is an essential cofactor for the c-Myc and E2F oncoproteins. Cell 94: 363-74.

Matsui, Y. (1998). Regulation of germ cell death in mammalian gonads. APMIS 106:142-147.

Miyazawa, A., Takahashi, M., Horikoshi, T. and Yoshioka, T. (1993). Immunohistochemical analysis of signal transduction system in developing rat Purkinje cell by using antibodies for signaling molecules. Ann N Y Acad Sci 707: 533-5.

Morgan J. I. Cohen, D. R. Hempstead, J. L. and Curran, T. (1987). Mapping patterns of c-fos expression in the central nervous system after seizure. Science 237: 192-7.

Morgan, J. I. and Curran, T. (1995). Immediate-early genes: ten years on. Trends Neurosci 18: 66-7.

Peterson, K., Rosenblum, M. K., Kotanides, H. and Posner, J. B. (1992). Paraneoplastic cerebellar degeneration. I. A clinical analysis of 55 anti-Yo antibody-positive patients. Neurology 42: 1931-1937.

Pietilainen, T., Lipponen, P., Aaltomaa, S., Eskelinen, M., Kosma, V. M. and Syrjanen, K. (1995). Expression of c-myc proteins in breast cancer as related to established prognostic factors and survival. Anticancer Res 15: 959-64.

Posner, J. B. (1995). Neurologic complications of cancer. (F. A. Davis Co., Philadelphia). Rao, A., Luo, C. and Hogan, P. G. (1997). Transcription factors of the NFAT family: regulation and function. Annu Rev Immunol 15: 707-47.

Royds, J. A., Sharrard, R. M., Wagner, B. and Polacarz, S. V. (1992). Cellular localisation of c-myc product in human colorectal epithelial neoplasia. J Pathol 166: 225-33.

Ruppert, C., Goldowitz, D. and Wille, W. (1986). Proto-oncogene c-myc is expressed in cerebellar neurons at different developmental stages. EMBO J 5: 1897-1901.

Sakai, K., Mitchell, D. J., Tsukamoto, T. and Steinman, L. (1990). Isolation of a complementary DNA clone encoding an autoantigen recognized by an anti-neuronal antibody from a patient with paraneoplastic cerebellar degeneration. Ann Neurol 28: 692-698.

Sakai, K., Ogasawara, T., Hirose, G., Jaeckle, K. A. and Greenlee, J. E. (11993). Analysis of auto antibody binding to 52-kd paraneoplastic cerebellar degeneration-associated antigen expressed in recombinant proteins. Ann Neurol 33: 373-80.

Sasano, H., Nagura, H. and Silverberg, S. G. (1992). Immunolocalization of c-myc oncoprotein in mucinous and serous adenocarcinomas of the ovary. Hum Pathol 23: 491-5.

Schmitz, M. L. and Baeuerle, P. A. (1995). Multi-step activation of NF-kappa B/Rel transcription factors. Immunobiology 193: 116-27.

Shrivastava, A., Saleque, S., Kalpana, G. V., Artandi, S., Goff, S. P. and Calame, K. (1993). Inhibition of transcriptional regulator Yin-Yang-1 by association with c-Myc. Science 262: 1889-92.

Takahashi, M., Toyoshima, S., Miyazawa, A., Horikoshi, T. and Yoshioka, T. (1993). Regulation of c-MYC protein expression in the developing rat cerebellum by phosphoinositide turnover. Biochem Biophys Res Commun 197: 278-86.

Takanaga, H., Mukai, H., Shibata, H., Toshimori, M. and Ono, Y. (1998). PKN interacts with a paraneoplastic cerebellar degeneration-associated antigen, which is a potential transcription factor. Exp Cell Res 241: 363-72.

Ulrich, E. Duwel, A., Kauffmann-Zeh, A., Gilbert, C., Lyon, D., Rudkin, B., Evan, G. and Martin-Zanca, D. (1998). Specific TrkA survival signals interfere with different apoptotic pathways. Oncogene 16: 825-32.

van Steensel, B. and de Lange, T. (1997). Control of telomere length by the human telomeric protein TRF1 [see comments]. Nature 385: 740-3.

Verschuuren, J., Chuang, L., Rosenblum, M. K., Lieberman, F., Pryor, A., Posner, J. B. and Dalmau, J. (1996). Inflammatory infiltrates and complete absence of Purkinje cells in anti-Yo-associated paraneoplastic cerebellar degeneration. Acta Neuropathol 91: 519-25.

Vriz, S., Lemaitre, J. M., Leibovici, M., Thierry, N. and Mechali, M. (1992). Comparative analysis of the intracellular localization of c-Myc, c-Fos, and replicative proteins during cell cycle progression. Mol Cell Biol 12: 3548-55.

Wakamatsu, Y., Watanabe, Y., Shimono, A. and Kondoh, H. (1993). Transition of localization of the N-Myc protein from nucleus to cytoplasm in differentiating neurons. Neuron 10: 1-9.

Wang, Y., Toury, R., Hauchecome, M. and Balmain, N. (1997). Expression and subcellular localization of the Myc superfamily proteins: c-Myc, Max, Mad1 and Mxi1 in the epiphyseal plate cartilage chondrocytes of growing rats. Cell Mol Biol (Noisy-le-grand) 43: 175-88.

What is claimed is:

1. A method for inducing apoptosis in cells of a mammal by administering a therapeutically effective amount of an agent capable of antagonizing the interaction between an onconeural antigen and an apoptosis-inducing protein.

2. The method of claim 1 wherein said cells are dysproliferative cells.

3. The method of claim 2 wherein said dysproliferative cells are cancer cells.

4. The method of claim 3 wherein said cancer is a gynecological cancer.

5. The method of claim 4 wherein said gynecological cancer is ovarian or breast cancer.

6. The method of claim 1 wherein said cells are normal cells.

7. The method of claim 6 wherein said normal cells are germ cells.

8. The method of claim 1 wherein said onconeural antigen is cdr2, cdr3, Nova, Hu, or amphiphysin.

9. The method of claim 1 wherein said onconeural antigen is cdr2.

10. The method of claim 1 wherein said apoptosis-inducing protein is a transcription factor.

11. The method of claim 10 wherein said transcription factor is N-Myc or C-myc.

12. The method of claim 1 wherein said agent is an antibody or antigen-binding fragment thereof.

13. The method of claim 12 wherein said antibody or antigen-binding fragment thereof binds to said onconeural antigen.

14. The method of claim 13 wherein said onconeural antigen is cdr2, cdr3, Nova, Hu, or amphiphysin.

15. The method of claim 13 wherein said antibody or antigen-binding fragment thereof binds to cdr2.

16. The method of claim 1 wherein said agent is an HLZ region-binding molecule.

17. The method of claim 16 wherein said agent is an HLZ region-binding polypeptide fragment of an onconeural antigen.

18. The method of claim 17 wherein said agent is an HLZ region-binding fragment of cdr2.

19. The method of claim 18 wherein said agent is a polypeptide comprising amino acids 16 through 192 of cdr2 (SEQ ID NO:1) or amino acids 65 through 140 of cdr2 (SEQ ID NO:2).

20. A method for treating a mammal suffering from a dysproliferative disease by administering a therapeutically effective amount of an agent capable of antagonizing the interaction between an onconeural antigen and an apoptosis-inducing protein.

21. The method of claim 20 wherein said dysproliferative disease is cancer.

22. The method of claim 21 wherein said cancer is a gynecological cancer.

23. The method of claim 22 wherein said gynecological cancer is ovarian or breast cancer.

24. The method of claim 20 wherein said onconeural antigen is cdr2, cdr3, Nova, Hu, or amphiphysin.

25. The method of claim 20 wherein said onconeural antigen is cdr2.

26. The method of claim 20 wherein said apoptosis-inducing protein is a transcription factor.

27. The method of claim 26 wherein said transcription factor is N-Myc or C-myc.

28. The method of claim 20 wherein said agent is an antibody or antigen-binding fragment thereof.

29. The method of claim 28 wherein said antibody or antigen-binding fragment thereof binds to said onconeural antigen.

30. The method of claim 29 wherein said onconeural antigen is cdr2, cdr3, Nova, Hu, or amphiphysin.

31. The method of claim 28 wherein said antibody or antigen-binding fragment thereof binds to cdr2.

32. The method of claim 20 wherein said agent is an HLZ region-binding molecule.

33. The method of claim 32 wherein said agent is an HLZ region binding polypeptide fragment of an onconeural antigen.

34. The method of claim 33 wherein said agent is an HLZ region-binding fragment of cdr2.

35. The method of claim 34 wherein said agent is a polypeptide comprising amino acids 16 through 192 of cdr2 (SEQ ID NO:1) or amino acids 65 through 140 of cdr2 (SEQ ID NO:2).

36. A method for identifying an agent capable of promoting apoptosis by antagonizing the interaction between an onconeural antigen and an apoptosis-inducing protein comprising the steps of
i) preparing a mixture comprising an onconeural antigen or a fragment thereof and an apoptosis-inducing protein or a fragment thereof, said mixture being part of a cell-free or cell-based test system;
ii) contacting said mixture with an agent being evaluated for its ability to antagonize the interaction between said onconeural antigen and said apoptosis-inducing protein;
iii) evaluating the extent of interference by said agent of the interaction between said onconeural antigen and said apoptosis-inducing protein; and
iv) determining from said extent of interference the capability of said agent to interfere with said interaction

37. The method of claim 36 wherein one or both of said onconeural antigen or a fragment thereof and said apoptosis-inducing protein or a fragment thereof additionally includes a detectable polypeptide sequence.

38. The method of claim 36 wherein said interaction is determined by assessing the decrease caused by said agent in the extent of binding of said onconeural antigen or a fragment thereof with said apoptosis-inducing protein or a fragment thereof.

39. The method of claim 38 wherein said extent of binding is determined using electrophoretic means.

40. The method of claim 38 wherein one of said onconeural antigen or apoptosis-inducing protein or fragments thereof is immobilized during the determination of said extent of interference.

41. The method of claim 38 wherein the extent of binding is determined in a GST pull-down assay.

42. The method of claim 38 wherein said extent of binding is determined in a coprecipitation assay.

43. The method of claim 38 wherein said extent of interference is determined by assessing the extent of transcriptional activity by said transcription factor.

44. The method of claim 36 wherein said extent of interference is determined using a whole cell assay and employing immunohistochemical means for quantitating the level of transcription factor in the subcellular compartments.

45. The method of claim 36 wherein said extent of interference is measured by quantitating cell death in a whole cell assay.

46. The method of claim 36 wherein said onconeural antigen is cdr2, cdr3, Nova, Hu, or amphiphycin.

47. The method of claim 36 wherein said onconeural antigen is cdr2.

48. The method of claim 36 wherein said apoptosis-inducing protein is a transcription factor

49. The method of claim 48 wherein said transcription factor is N-Myc or C-myc.
